(19) United States
(12) Patent Application Publication
(10) Pub. No.: US 2015/0223965 A1
(43) Pub. Date: Aug. 13, 2015

(54) SLEEPING AID NASAL BAND

(71) Applicant: Sundex Corporation, New Taipei City (TW)

(72) Inventor: Jan-Mao Chen, New Taipei City (TW)

(21) Appl. No.: 14/517,809

(22) Filed: Oct. 18, 2014

(30) Foreign Application Priority Data
Feb. 13, 2014 (TW) 103202496

(51) Int. Cl. A61F 5/08

(52) U.S. Cl. CPC A61F 5/08

(57) ABSTRACT

A sleeping aid nasal band, comprising: a carrier and a water absorption material. On one side of the carrier is provided with an attachment area. The water absorption material is disposed on the carrier, while a volatile material is placed in the water absorption material. In application, through the volatile material placed in the water absorption material, the user is relieved of tension, to fall into a nice and sound sleep easily. Therefore, the sleeping aid nasal band is able to reduce cost, while achieving convenience in usage.

### BACKGROUND OF THE INVENTION

1. Field of the Invention

The present invention relates to a sleeping aid nasal band, and in particular to a sleeping aid nasal band formed by a carrier and a water absorption material.

2. The Prior Arts

Along with the rapid progress and development of business and industry in the modern age, most people keep a quick tempo life style and busy work schedule, so that the pressure of work and life is increasing. As such, even after work, people may still feel tension for the pressure of work, thus leading to the problem of sleeplessness. In this respect, in case adequate sleep is not obtained for a long period of time, that could cause people to become drowsy, dizzy, irritable, and getting slow in motion, as such even leading to various chronic diseases.

To redress this problem, various researches have been conducted to develop medicine and device to relieve the problem of sleeplessness. Among the various approaches presently available, the essence fumigation is most acceptable. The principle of its approach is that, through applying massage using essence oil and through smelling scent of the essence oil, muscle and mental tension can be relieved, to reduce pressure of work. Also, in this approach, it can make people to have a feeling of soothing and tranquility, thus improving the mood and mentality of people, in achieving reducing tension, anxiety, and pressure. Therefore, essence fumigation is very effective in aiding people to fall into sleep.

However, the equipment required for essence fumigation is quite numerous, such as fumigation table, heater, and essence oil. In addition, the space required for its application constitutes a restriction. The reason for this is that, in case it is applied in a large space, people may not be able to fall into sleep through smelling the scent of the essential oil, since the scent is dissipated to thin in a large space, and its effect is reduced. Besides, in performing essence fumigation, good air convection is essential.

Therefore, presently, for a sleeping aid device, the problem of how to increase convenience in application, be suitable to use on various parts of the body, while achieving good sleeping aid effect, is an important task that has to be solved in this field.

### SUMMARY OF THE INVENTION

A major objective of the present invention is to provide a sleeping aid nasal band, formed by a carrier and a water absorption material, to aid people to fall into sleep easily, while realizing convenience in application and reduction of cost.

In order to achieve the objective mentioned above, the present invention provides a sleeping aid nasal band, formed by a carrier and a water absorption material. On one side of the carrier is provided with an attachment area. The water absorption material is placed in the carrier, while a volatile material is disposed in the water absorption material.

In an aspect of the present invention, the carrier is made of porous plastic material.

In another aspect of the present invention, the carrier is provided with an adhesive layer, disposed in the attachment area.

In a yet another aspect of the present invention, the carrier is made of biological fiber.

In a further aspect of the present invention, the carrier is made of natural fiber.

In a yet another aspect of the present invention, the carrier is provided with a plurality of through holes.

In another aspect of the present invention, the water absorption material is formed by fabric or plastic complex.

In a further aspect of the present invention, the water absorption material is formed by non-woven cloth.

In another aspect of the present invention, the volatile material is essential oil.

In a yet another aspect of the present invention, the volatile material is flavor essence.

Further scope of the applicability of the present invention will become apparent from the detailed descriptions given hereinafter. However, it should be understood that the detailed descriptions and specific examples, while indicating preferred embodiments of the present invention, are given by way of illustration only, since various changes and modifications within the spirit and scope of the present invention will become apparent to those skilled in the art from this detailed descriptions.

### BRIEF DESCRIPTION OF THE DRAWINGS

The related drawings in connection with the detailed descriptions of the present invention to be made later are described briefly as follows, in which:

FIG. 1 is a top view of a sleeping aid nasal band according to a first embodiment of the present invention;

FIG. 2 is a cross section view of a sleeping aid nasal band according to a first embodiment of the present invention;

FIG. 3 is a top view of a sleeping aid nasal band according to a second embodiment of the present invention; and

FIG. 4 is a cross section view of a sleeping aid nasal band according to a second embodiment of the present invention;

FIG. 5 is a top view of a sleeping aid nasal band according to a third embodiment of the present invention; and

FIG. 6 is a cross section view of a sleeping aid nasal band according to a third embodiment of the present invention.

### DETAILED DESCRIPTION OF THE PREFERRED EMBODIMENT

The purpose, construction, features, functions and advantages of the present invention can be appreciated and understood more thoroughly through the following detailed description with reference to the attached drawings.

### The First Embodiment

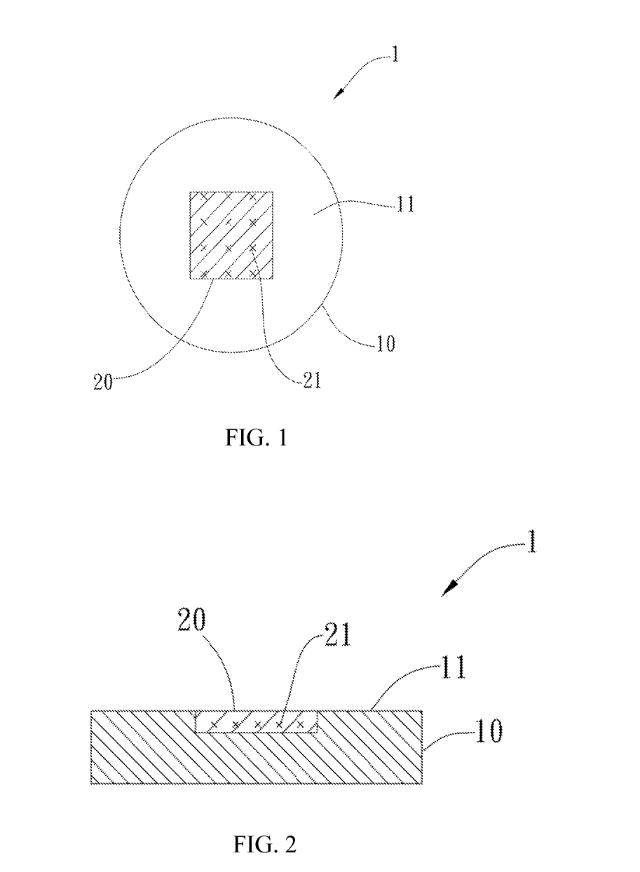

Refer to FIGS. 1 and 2 respectively for a top view of a sleeping aid nasal band according to a first embodiment of the present invention, and a cross section view of a sleeping aid nasal band according to a first embodiment of the present invention. As shown in FIGS. 1 and 2, a sleeping aid nasal band 1 is formed by a carrier 10 and a water absorption material 20. On one side of the carrier 10 is provided with an attachment area 11. The water absorption material 20 is placed on the carrier 10, while a volatile material 21 is disposed in the water absorption material 20.

In the present embodiment, the carrier 10 is formed by non-woven cloth made of natural fiber. The water absorption material 20 is formed by fabric or plastic complex. However, the present invention is not limited to this. In practice, the carrier 10 can be formed by other material, such as knitted cloth or woven fabric made of biologic fiber or synthetic fiber. For the water absorption material 20, such as the non-woven cloth having water absorption capability or other relative absorption material can also be utilized.

Refer again to FIG. 1 for a top view of a sleeping aid nasal band according to a first embodiment of the present invention. As shown in FIG. 1, in the application of the first embodiment, firstly, place the attachment area 11 of the carrier 10 to contact water, to make it become soft, and then put the volatile material 21 into the water absorption material 20. Finally, place the attachment area 11 of the carrier 10 close to the nose (alternatively, the volatile material 21 can be put into the water absorption material 2 in advance for packaging, so that it only requires to make the attachment area 11 of the carrier 10 to become soft to be applicable). In this way, the user may smell the scent given out by the volatile material 21, in realizing the relaxing and sleeping aid effect. In the present embodiment, the adhesive tape approach is used to save cost. In addition, it has the benefit that, the sleeping aid nasal band 1 can be used on various parts of user's body, to achieve convenience in usage. In the present embodiment, the volatile material 21 can be selected from natural essential oil or synthetic essential oil (for example, rose essential oil, geranium essential oil, teaseed essential oil, cypress essential oil, terebinth essential oil, frankincense essential oil, mugwort essential oil, and lavender essential oil). However, the present invention is not limited to this. In practice, the volatile material 21 can also selected from natural flavor essence or synthetic flavor essence.

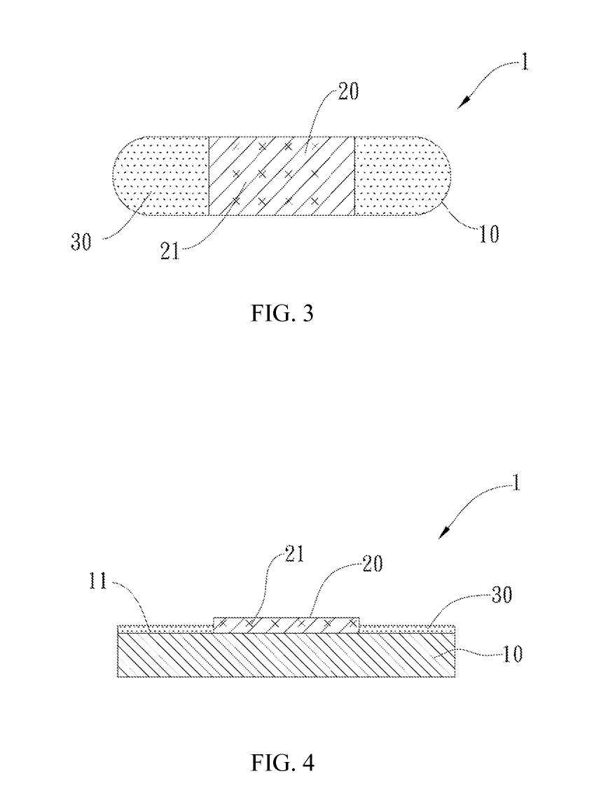

For convenience of explanations, similar elements in FIGS. 3 to 6 utilize the same numerals as in FIGS. 1 and 2.

### The Second Embodiment

Refer to FIGS. 3 and 4 respectively for a top view of a sleeping aid nasal band according to a second embodiment of the present invention, and a cross section view of a sleeping aid nasal band according to a second embodiment of the present invention. As shown in FIGS. 3 and 4, a sleeping aid nasal band 1 is formed by a carrier 10 and a water absorption material 20. On one side of the carrier 10 is provided with an attachment area 11. The water absorption material 20 is placed in the carrier 10, while a volatile material 21 is disposed in the water absorption material 20.

In the present embodiment, the carrier 10 is made of plastic material, and an adhesive layer 30 is placed in the attachment area 11. The water absorption material 20 is formed by fabric or plastic complex. However, the present invention is not limited to this. In practice, the carrier 10 can be made of other suitable materials, while the water absorption material 20 can be formed by non-woven cloth having water absorption capability.

Refer again to FIG. 3 for a top view of a sleeping aid nasal band according to a second embodiment of the present invention, in this embodiment, the sleeping aid nasal band is made into a form of bandages. In application, firstly, place the volatile material 21 into the water absorption material 20, then place and attach the adhesive layer 30 of the carrier 10 to the place close to the nose (alternatively, the volatile material 21 can be put into the water absorption material 20 in advance for packaging, such that it only requires to attach the adhesive layer 30 of the carrier 10 to the place close to the nose to be applicable). In this way, the user may smell the scent given out by the volatile material 21, in realizing the relaxing and sleeping aid effect. In the present embodiment, the adhesive tape approach is used to save cost. In addition, it has the benefit that, the sleeping aid nasal band can be used everywhere, to achieve convenience in usage. In the present embodiment, the volatile material 21 can be selected from natural essential oil or synthetic essential oil (for example, rose essential oil, geranium essential oil, teaseed essential oil, cypress essential oil, terebinth essential oil, frankincense essential oil, mugwort essential oil, and lavender essential oil). However, the present invention is not limited to this. In practice, the volatile material 21 can also selected from natural flavor essence or synthetic flavor essence.

### The Third Embodiment

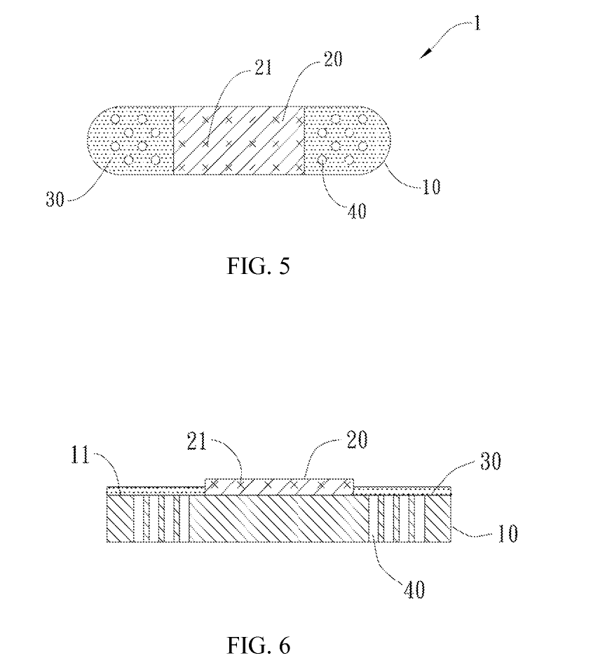

Refer to FIGS. 5 and 6 respectively for a top view of a sleeping aid nasal band according to a third embodiment of the present invention, and a cross section view of a sleeping aid nasal band according to a third embodiment of the present invention. As shown in FIGS. 5 and 6, a sleeping aid nasal band 1 is formed by a carrier 10 and a water absorption material 20. On one side of the carrier 10 is provided with an attachment area 11. The water absorption material 20 is placed in the carrier 10, while a volatile material 21 is disposed in the water absorption material 20.

In the present embodiment, the carrier 10 is made of plastic material, and an adhesive layer 30 is placed in the attachment area 11. The adhesive layer 30 is provided with a plurality of through-holes 40 to penetrate through the carrier 10, while the water absorption material 20 formed by fabric or plastic complex. However, the present invention is not limited to this. In practice, the carrier 10 can be made of other suitable materials, while the water absorption material 20 can be formed by non-woven cloth having water absorption capability.

Refer again to FIG. 5 a top view of a sleeping aid nasal band according to a third embodiment of the present invention. In this embodiment, the way of application is the same as that of the second embodiment. The difference is that, in the present embodiment, the carrier 10 is provided with a plurality of through-holes 40 to penetrate through the carrier 10. The advantages of this type of design is to provide ventilation via the through-holes, to avoid swollen skin caused by over sensitiveness due to poor ventilation.

The above detailed description of the preferred embodiment is intended to describe more clearly the characteristics and spirit of the present invention. However, the preferred embodiments disclosed above are not intended to be any restrictions to the scope of the present invention. Conversely, its purpose is to include the various changes and equivalent arrangements which are within the scope of the appended claims.

What is claimed is:

1. A sleeping aid nasal band, comprising:
a carrier, on its one side is provided with an attachment area; and
a water absorption material, disposed in said carrier, and a volatile material is placed in said water absorption material.

2. The sleeping aid nasal band as claimed in claim 1, wherein said carrier is made of plastic material.

3. The sleeping aid nasal band as claimed in claim 1, wherein on said carrier is provided with an adhesive layer, that is disposed in an attachment area.

4. The sleeping aid nasal band as claimed in claim 1, wherein said carrier is made of biological fiber.

5. The sleeping aid nasal band as claimed in claim 1, wherein said carrier is made of natural fiber.

6. The sleeping aid nasal band as claimed in claim 1, wherein said carrier is provided with a plurality of through-holes.

7. The sleeping aid nasal band as claimed in claim 1, wherein said water absorption material is formed by fabric or plastic complex.

8. The sleeping aid nasal band as claimed in claim 1, wherein said water absorption material is formed by non-woven cloth.

9. The sleeping aid nasal band as claimed in claim 1, wherein said volatile material is natural essential oil or synthetic essential oil.

10. The sleeping aid nasal band as claimed in claim 1, wherein said volatile material is natural flavor essence or synthetic flavor essence.
